(19) United States
(12) Patent Application Publication
(10) Pub. No.: US 2014/0248214 A1
(43) Pub. Date: Sep. 4, 2014

(54) High-concentration aqueous dispersions of graphene using nonionic, biocompatible copolymers

(71) Applicant: Northwestern University, Evanston, IL (US)

(72) Inventors: Mark C. Hersam, Wilmette, IL (US); Jung-Woo T. Seo, Evanston, IL (US); Alexander A. Green, Boston, MA (US); Alexander L. Antaris, Evanston, IL (US)

(21) Appl. No.: 13/815,835

(22) Filed: Mar. 14, 2013

Related U.S. Application Data
(60) Provisional application No. 61/623,465, filed on Apr. 12, 2012.

(51) Int. Cl. A61K 47/02; A61K 47/10; A61K 49/00

(52) U.S. Cl. CPC A61K 47/02; A61K 49/0002; A61K 47/10
USPC 424/9.1; 514/769

(57) ABSTRACT

Methods of using a surface active block copolymer to disperse graphene in an aqueous medium, such dispersions which can be subsequently separated and processed for a range of end-use applications, including biomedical applications.

This application claims priority benefit from application Ser. No. 61/623,465 filed Apr. 12, 2012, the entirety of which is incorporated herein by reference.

This invention was made with government support under grant numbers DMR0520513, EEC0647560 and DMR1006391 awarded by the National Science Foundation and grant number W911NF-05-1-0177 awarded by the Army Research Office. The government has certain rights in the invention.

### BACKGROUND OF THE INVENTION

Graphene exhibits a number of exceptional properties that make it a promising material for use in biological systems. Its high surface area, hydrophobicity, and nanometer-scale thickness can be exploited to deliver low-solubility drugs to cells, target tumors, and enable biological imaging. Furthermore, the strong near-infrared optical absorption of graphene provides a pathway to eliminating malignant cells through photothermal ablation. An enabling step in these applications is the development of methods to suspend graphene at high concentrations in aqueous solutions using biocompatible dispersing agents. Prior work has shown that stable suspensions of graphene oxide can be readily produced in water and in a number of organic solvents. This chemically modified graphene can subsequently be reduced to regain some of the properties of pristine graphene while being stabilized in aqueous solution with biocompatible polymers. Although high concentrations of reduced graphene oxide can be obtained using this approach, harsh chemical treatments are typically employed to both oxidize and reduce the graphene, which complicates processing, reduces compatibility with living systems, and raises concerns over its long-term environmental impact.

Alternatively, stable pristine graphene dispersions can be obtained directly from pristine graphite sources using organic solvents, superacids, and aqueous solutions containing amphiphilic surfactants. Whereas these approaches obviate the need for aggressive chemical functionalization, the use of organic solvents, superacids, and ionic surfactants for dispersion generally precludes their use in biological systems. Moreover, only a limited number of these systems have been shown to exfoliate pristine graphene at useful concentrations. Consequently, there remains an on-going search in the art for one or more dispersing agents capable of efficiently exfoliating and stabilizing pristine graphene in aqueous solution.

### SUMMARY OF THE INVENTION

In light of the foregoing, it is an object of the present invention to provide one or more methods, systems and/or compositions relating to graphene dispersions and preparation thereof, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative to any one aspect of this invention.

It can be an object of the present invention to provide a range of surface active copolymers that can be rationally designed and tailored to control and/or enhance dispersion of graphene in aqueous media.

It can be an object of this invention to provide a class of biocompatible dispersing agents for graphene in aqueous media as a step toward large-scale processing of the sort required for emerging end-use applications.

It can be another object of this invention to provide aqueous graphene dispersions at cost-effective concentrations.

It can be another object of the present invention, alone or in conjunction with one or more of the preceding objectives, to provide stable, high-concentration graphene dispersions with graphene nanoplatelets dimensioned to reduce cytotoxicity, for use in a range of biomedical applications.

Other objects, features, benefits and advantages of the present invention will be apparent from this summary and the following descriptions of certain embodiments, and will be readily apparent to those skilled in the art knowledgeable regarding graphene dispersions, use and properties. Such objects, features, benefits and advantages will be apparent from the above as taken into conjunction with the accompanying examples, data, figures and all reasonable inferences to be drawn therefrom, alone or with consideration of one or more references incorporated herein.

In part, this invention can be directed to a method of preparing an aqueous graphene dispersion. Such a method can comprise providing a system comprising an a aqueous fluid medium, a graphitic composition comprising natural graphene, and an amphiphilic surface active polymeric component, comprising a poly(ethylene oxide) group; applying waveform energy to and/or sonicating such a system for a time and/or at an energy sufficient to at least partially exfoliate a graphene component and disperse it within such a fluid medium; and centrifuging such a sonicated system for a time and/or rotational rate at least partially sufficient to separate such a graphene component from undispersed graphitic material. The dispersed graphene can be analyzed spectrophotometrically to determine concentration, and deposited films can be examined microscopically to characterize corresponding graphene platelets in terms of thickness dimension and layer number.

The graphene component can be provided in composition with a nonionic, poly(ethylene oxide)-containing polymer of the sort understood by those skilled in the art made aware of this invention. Generally, such a polymer component can function, in conjunction with a particular fluid medium, to exfoliate and stabilize graphene. In certain embodiments, such a component can be selected from a wide range of nonionic amphiphiles. In certain non-limiting embodiments, such a polymeric component can comprise a relatively hydrophilic poly(ethylene oxide) (PEO) group and a relatively hydrophobic moiety. In certain other non-limiting embodiments, such a component can be selected from various linear block poly(alkylene oxide) copolymers. In certain such embodiments, such poly(alkylene oxide) copolymer components can be X-shaped and/or coupled with a linker such as but not limited to an alkylene diamine moiety. Regardless, without limitation, such copolymer components can comprise PEO and poly(propylene oxide) (PPO) blocks, as discussed more fully, below. More generally, such embodiments are representative of a broader group of polymeric surface active components capable of providing a structural configuration about and upon interaction with graphene platelets in a fluid medium.

In part, the present invention can also be directed to a method of using a surface active block copolymeric component to affect dispersion of graphene in an aqueous medium. Such a method can comprise providing a system comprising an aqueous fluid medium, a graphene source material comprising a graphene component, and at least one surface active block copolymeric component comprising a poly(alkylene oxide) block; exfoliating such a graphene component; and centrifuging the system for a time and/or at a rotational rate at least partially sufficient to separate such a graphene component from undispersed material. Useful fluid medium and surface active components, can be as described elsewhere herein.

Regardless, such a block copolymeric component can be of the sort discussed herein and/or illustrated more fully below. In certain such embodiments, such a component can comprise hydrophilic and hydrophobic poly(alkylene oxide) blocks. Without limitation, whether or not coupled by an alkylene diamine linker moiety, such copolymer components can comprise hydrophilic PEO and hydrophobic PPO blocks. In certain such embodiments, exfoliation and/or dispersion can be enhanced by increasing the molecular weight of the hydrophilic blocks (e.g., up to about 30-about 90 wt % or up to about 60-about 90 wt. %), up to a certain overall molecular weight. In certain non-limiting embodiments, such a copolymer can be selected from Pluronics F68, F77, and F87, and Tetronics 1107 and 1307—copolymers comprising about 70-about 80 percent PEO by weight.

In part, this invention can be directed to a method of using a density gradient to separate graphene. Such a method can comprise providing a fluid medium comprising a density gradient; contacting such a medium and a composition comprising graphene source material and a surface active block copolymeric component of the sort discussed above, sonicated as described herein and dispersed in an aqueous medium; and centrifuging the medium and graphene dispersion for a time and/or rotational rate at least partially sufficient to separate the graphene along a medium gradient. The graphene selectively separated and/or isolated by platelet thickness dimension and/or layer number can be identified spectrophotometrically and/or assessed by concentration, such a concentration enriched relative to an foregoing dispersion.

Fluid media useful with a centrifugation/separation aspect of this invention are limited only by graphene aggregation therein to an extent precluding at least partial separation. Accordingly, without limitation, aqueous and non-aqueous fluids can be used in conjunction with any substance soluble or dispersible therein, over a range or with a plurality of concentrations so as to provide the medium a density gradient for use in the separation techniques described herein. Such substances can be ionic or non-ionic, non-limiting examples of which include inorganic salts and alcohols, respectively. In certain embodiments, as illustrated more fully below, such a medium can comprise a plurality and/or range of aqueous iodixanol concentrations and a corresponding gradient of concentration densities. Likewise, the methods of this invention can be influenced by gradient slope, as affected by length of centrifuge compartment and/or angle of centrifugation.

Regardless of medium identity or density gradient, contact can comprise introducing one or more of the aforementioned graphene dispersions on or at any point within the gradient, before centrifugation. In certain embodiments, such a dispersion can be introduced at a position along the gradient which can be substantially invariant over the course of centrifugation. Such an invariant point can be advantageously determined to have a density corresponding to about or approximating the buoyant density of the graphene dispersion(s) introduced thereto.

Upon sufficient centrifugation, at least one fraction of the medium or graphene dispersion can be separated and/or isolated from the medium, such fraction(s) as can be isopycnic at a position along the gradient. Any such medium and/or graphene fraction can be used, or optionally reintroduced to another fluid medium, for subsequent refinement or separation. Accordingly, such a method of this invention can comprise repeating or iterative centrifuging, separating and isolation. In certain embodiments, medium conditions or parameters can be maintained from one separation to another. In certain other embodiments, however, at least one iterative separation can comprise a change of one or more parameters, such as but not limited to the identity of the surface active component(s), medium identity, medium density gradient and/or various other medium parameters with respect to one or more of the preceding separations.

In part, the present invention can also be directed to a method of using a nonionic block copolymer to reduce graphene cytotoxicity. Such a method can comprise providing a system comprising an aqueous medium, a graphitic composition comprising a natural graphene component and an amphiphilic surface active polymeric component comprising a poly(ethylene oxide) block; and exfoliating such a graphene component to disperse it within such an aqueous medium. Resulting dispersed graphene platelets can have a thickness dimension less than about 10 nm. In certain such embodiments, platelet thickness can be less than about 4 nm. Regardless, with a lateral dimension from about 50 nm, up to about 250 nm or up to about 500 nm, such platelets can have an aspect ratio of about 1. Useful fluid medium and block copolymer components can be of the sort discussed herein and/or illustrated more fully below.

In part, the present invention can be directed to a graphene composition. Such a composition can comprise graphene nanoplatelets and an amphiphilic surface active block copolymeric component comprising a poly(ethylene oxide) block in an aqueous medium. Such a copolymeric component can be bound, coupled to, complexed or otherwise interactive with graphene. Such a composition can comprise a graphene concentration greater than about 0.07 mg/mL. Alternatively, such a composition can be characterized as a stable dispersion of graphene in an aqueous medium with an optical density greater than about 4 OD/cm. In the context of such a composition, the term “stable” can refer to the capacity of such a block copolymer to inhibit nanoplatelet aggregation of the sort precluding optical density measurement.

### BRIEF DESCRIPTION OF THE DRAWINGS

The patent or application file contains a least one drawing executed in color. Copies of this patent or patent application publication with color drawing(s) will be provided by the Office upon request and payment of the necessary fee.

FIG. 1. Chemical structures of Pluronic® and Tetronic® block copolymers.

FIGS. 2A-B. Schematic illustrations of the interaction of (A) Pluronic® and (B) Tetronic® block copolymers with graphene nanoplatelets.

FIGS. 3A-C. (A) Digital images of aqueous graphene dispersions in Pluronics® L64 and F77 and Tetronics® 904 and 1107. (B) Optical absorbance spectra of the copolymer graphene dispersions shown in panel A. (C) Graphene concentration map for Pluronics and Tetronics. Colored circles and squares represent the actual experimental graphene concentrations obtained for the Pluronic® and Tetronic® copolymers, respectively, whereas the underlying color map was obtained by averaging a moving window over the experimental Pluronic data.

FIGS. 4A-D. (A,B) SEM images of restacked graphene films produced using (A) Pluronic® F77 and (B) Tetronic® 1107. (C,D) AFM images of graphene nanoplatelets in (C) Pluronic F77 and (D) Tetronic 1107 deposited on SiO2. (D, bottom) AFM line profiles of graphene nanoplatelets. Scale bars: (A C) 500 and (D) 250 nm.

FIG. 4E. SEM images of graphene films obtained from dispersions using different block copolymers. The top three rows were produced using Pluronics and the lowest row was produced using Tetronics. The scale bar in all images is 500 nm.

FIGS. 5A-B. (A) Raman spectra at a 514 nm excitation wavelength obtained from restacked graphene films produced using Pluronic® F77 and Tetronics® 904 and 1107. (B) Graphene D/G ratio map for Pluronics and Tetronics. Colored circles and squares represent the actual experimental D/G ratios obtained for the Pluronic and Tetronic copolymers, respectively, while the underlying color map was obtained by averaging a moving window over the experimental Pluronic data.

FIG. 5C. Representative Raman spectrum in the G and D region for a graphene film obtained using Tetronic® 1107 along with corresponding fitting curves.

FIG. 6. Optical absorbance spectra of graphene nanoplatelet dispersions exfoliated and encapsulated by Tetronic® T1307. Significant enhancement in the attainable optical density of the stable dispersions is evident as a function of increasing sonication time.

FIGS. 7A-C. Isopycnic point-based DGU (i-DGU) of surfactant-encapsulated graphene nanoplatelets. (A) Scheme of i-DGU where two-dimensional nanomaterials travel towards their isopycnic points under ultracentrifugation. Thinner platelets have lower buoyant densities, thus they will be found at the top of the centrifuge tube following i-DGU. (B) i-DGU was utilized in a previous study (digital image) to separate sodium cholate-encapsulated GNS by layer number. (Prior Art). (C) A digital image showing similar banding behavior is observed with Tetronic® (T1307)-encapsulated nanoplatelets when subjected to an i-DGU protocol of the sort described below, in accordance with certain non-limiting embodiments of this invention.

### DETAILED DESCRIPTION OF CERTAIN EMBODIMENTS

Several non-limiting methods, systems and compositions were used to illustrate various aspects of this invention. A set of nonionic biocompatible copolymers, Pluronice® and Tetronic®-type block copolymers, were evaluated for their ability to disperse pristine graphene in aqueous solutions. Resulting graphene suspensions were found to have concentrations exceeding 0.07 mg mL⁻¹, which correspond to optical densities exceeding 4 OD cm⁻¹ in the visible and near-infrared regions of the electromagnetic spectrum. Scanning electron (SEM) and atomic force microscopy (AFM) indicate that the suspended graphene nanoplatelets have lateral dimensions of several hundred nanometers and thicknesses ranging from 1 to 10 graphene layers. A comprehensive survey of 19 representative Pluronic® and Tetronic® copolymers quantifies the effect of the hydrophobic and hydrophilic domain size on the concentration and defect density of the suspended graphene nanosheets.

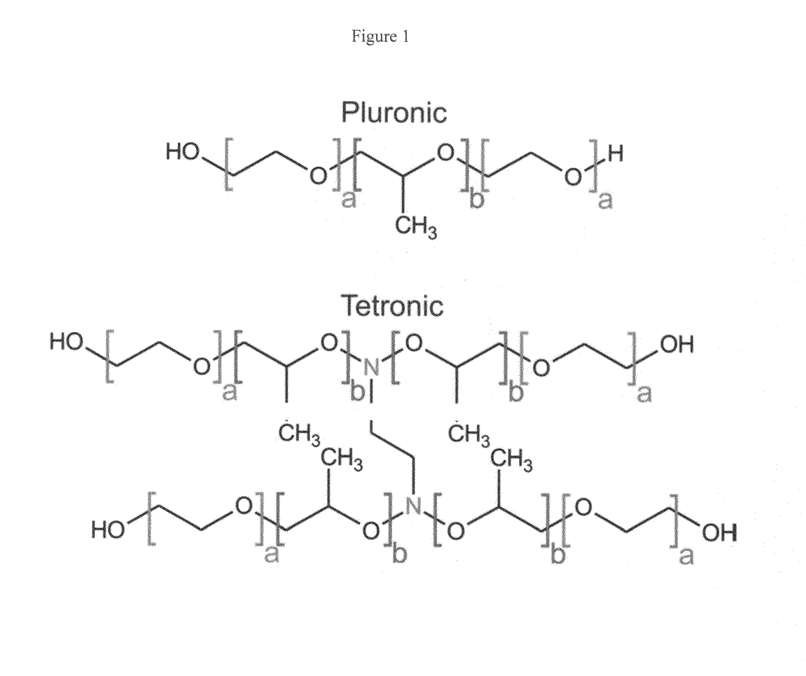

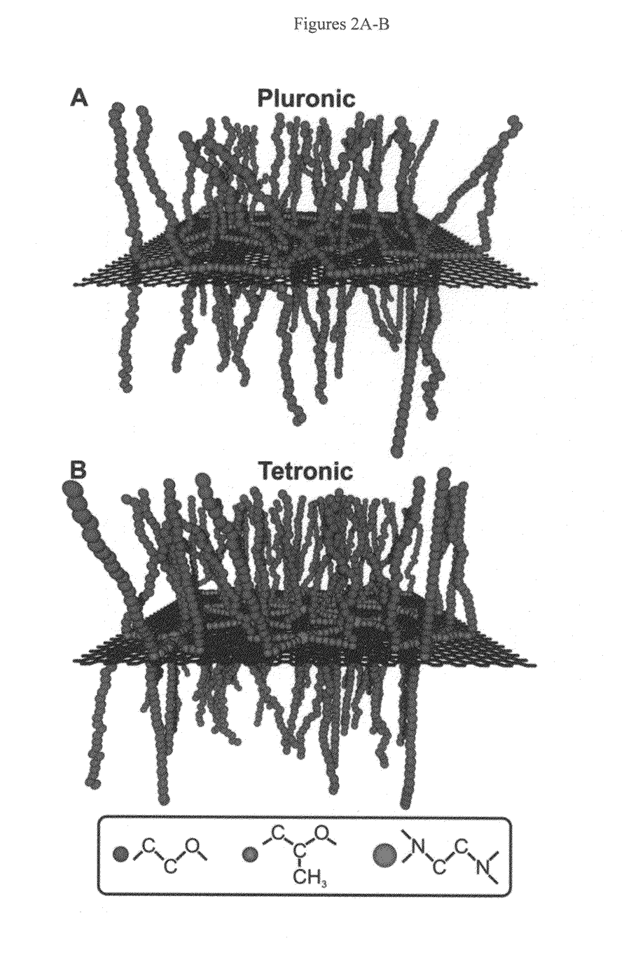

Pluronic® and Tetronic® polymers are commercially available nonionic, amphiphilic block copolymers containing hydrophobic polypropylene oxide (PPO) and hydrophilic polyethylene oxide (PEO) domains. Pluronics are linear molecules consisting of a central PPO region flanked on either end by PEO domains of equal length (FIG. 1 and FIG. 2A). In contrast, Tetronics are cross-shaped molecules containing a central ethylenediamine linker tethered to four identical diblock copolymer segments (FIG. 1 and FIG. 2B). These diblock segments consist of a PEO and PPO domain with the hydrophobic segment covalently bound to the nitrogen atoms of the linker. As demonstrated, the sizes of the hydrophobic and hydrophilic blocks of both Pluronics and Tetronics can be tuned independently, thereby providing a large number of possible copolymers to be tested for their effectiveness in dispersing graphene.

As understood in the art, both copolymers are conveniently named following the relative composition of their polymer blocks. The names of Pluronics begin with a letter that designates their state at room temperature (flake, paste, or liquid), followed by a set of two or three digits. The last of these digits multiplied by 10 denotes the percentage by weight of the PEO block, whereas the earlier digits multiplied by 300 correspond to the approximate average molecular weight of the PPO block. For example, Pluronic F68 exists in flake form at room temperature, consists of 80% PEO by molecular weight, and contains a PPO block with approximate molecular weight of 1800 Da. Tetronics follow a similar naming convention in which the last digit of their name multiplied by 10 designates the percentage by weight of their hydrophilic segments, whereas the earlier digits multiplied by 45 provide the approximate molecular weight of the PPO block. Without limitation to any one theory or mode of operation, in graphene suspensions, the hydrophobic PPO segments are believed to interact strongly with the graphene faces leaving the hydrophilic PEO chains free to interface with other nearby PEO chains and the surrounding aqueous environment (FIG. 2).

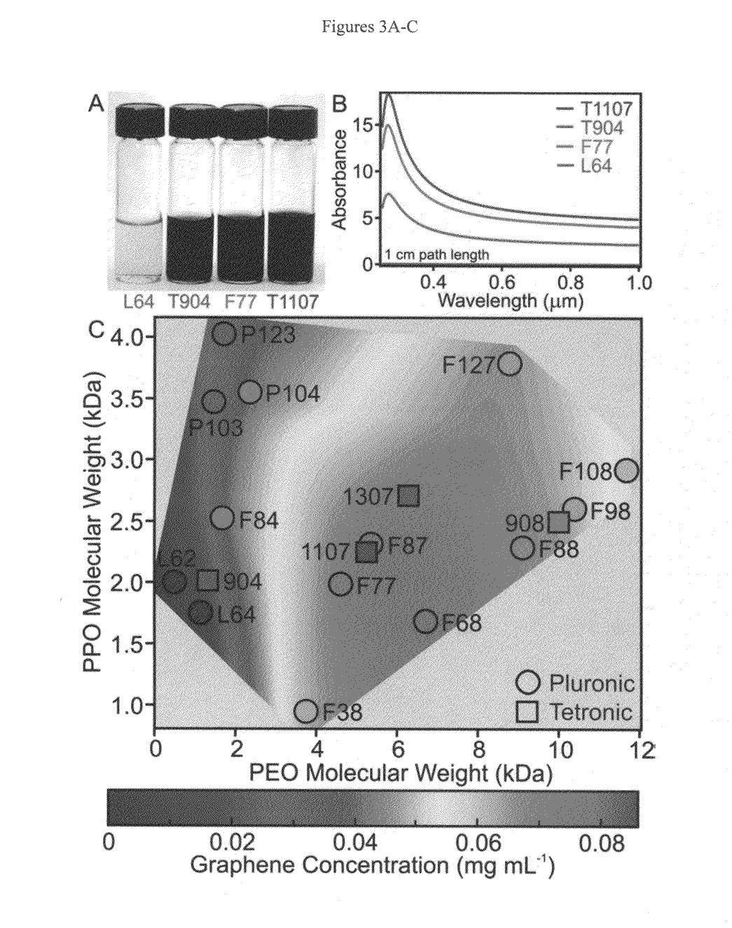

To prepare the graphene dispersions, 0.6 g of natural graphite flakes (Asbury Carbons, 3061 graphite) were combined with 8 mL of 1% w/v aqueous solution containing the block copolymer. (See Examples, below.) A horn ultrasonicator was used to exfoliate graphene directly from the graphite flakes through cavitation. The sonicated mixture was subsequently centrifuged to remove any poorly dispersed graphitic material. FIG. 3A displays graphene suspensions obtained using four different copolymers—illustrating various degrees of dispersion efficiency. For present purposes, the term “dispersion efficiency” describes the capacity of the block copolymer to produce stable graphene dispersions with relatively high concentrations. This parameter appears to be a function of the exfoliation efficiency (i.e., copolymer ability to tease apart neighboring graphene sheets) and stabilization efficiency (i.e., copolymer capacity for preventing individualized graphene sheets from reaggregating once exfoliated). The results of FIG. 3A show that small-molecular-weight Pluronics having predominantly hydrophobic composition, such as L64 and L62, were the least effective dispersing agents. In contrast, other copolymers, such as Pluronic® F77 and Tetronic® 1107, yielded dark black graphene dispersions.

To quantify dispersion efficiency, the optical absorbance of the graphene suspensions was measured in the ultraviolet, visible, and near-infrared regions of the electromagnetic spectrum (FIG. 3B). Those graphene dispersions with measurable optical absorbance displayed a strong peak at ˜268 nm, believed to arise from the π-plasmon resonance commonly observed in graphitic materials. (See, Eberlein, T.; Bangert, U.; Nair, R. R.; Jones, R.; Gass, M.; Bleloch, A. L.; Novoselov, K. S.; Geim, A.; Briddon, P. R. Plasmon Spectroscopy of Free-Standing Graphene Films. Phys. Rev. B 2008, 77, 233406.) For longer wavelengths, the absorption spectrum is featureless out to the near-infrared with a monotonic decrease in intensity with increasing wavelength. For the Pluronic L64 dispersion, the optical absorption of graphene was barely detectable, whereas the optical absorption increased progressively in the order: Tetronic 904, Pluronic F77, and Tetronic 1107.

To better understand the effect of PPO and PEO chain lengths, the dispersion efficiency was calculated for a set of 14 different Pluronic® and 5 different Tetronic® block copolymers. Graphene concentrations were determined from optical absorbance measurements using Beer's Law based on an extinction coefficient of 6600 L g⁻¹ m⁻¹. (See, Lotya, M.; King, P. J.; Khan, U.; De, S.; Coleman, J. N. High-Concentration, Surfactant-Stabilized Graphene Dispersions. ACS Nano 2010, 4, 3155-3162.) This extinction coefficient is believed to be the highest reported for graphene and was chosen to establish conservative lower bounds for the graphene concentrations dispersed by the block copolymers. (Experimental optical density values are tabulated in Table 2, below.) FIG. 3C summarizes the experimental data, plotting the resulting graphene loadings of all tested copolymers as a function of their hydrophilic and hydrophobic molecular weights. Colored circles and squares are used to represent the actual experimental graphene concentrations obtained for the Pluronic and Tetronic copolymers, respectively, whereas the underlying color map was determined by averaging a moving window over the experimental Pluronic data. (See, Examples, below.) In addition, the PEO and PPO molecular weights of the Tetronic polymers are plotted at half their actual values because Tetronics can be viewed as a pair of Pluronic chains connected at their midpoints.

Analysis of these results reveals two principal trends in the dispersion efficiency of the Pluronic® family. First, graphene nanoplatelets appear more efficiently exfoliated as the molecular weight of the PEO block size increases. Similar to effects observed with carbon nanotubes, it is likely that Pluronics having short PEO segments do not provide sufficient steric hindrance to prevent nearby graphene platelets from interacting and ultimately aggregating with one another in solution. Second, Pluronic copolymers sharing the same percentage molecular weight of PEO exhibit dispersion efficiencies that peak at a particular overall molecular weight. This effect is most clearly observed in Pluronics F38, F68, F88, F98, and F108 in FIG. 3C, which all are 80% PEO by molecular weight. Without limitation, this phenomenon likely arises as a result of two countervailing forces. On the one hand, the hydrophobic domain of the copolymer must be large enough to interface strongly with the graphene to separate it from its neighbors. On the other hand, copolymers having very high molecular weights are too bulky to intercalate between graphene layers for efficient exfoliation. The above trends lead to a various dispersion embodiments preferably using Pluronics F68, F77, and/or F87.

Because there are fewer members of the Tetronic® copolymer family, the survey of their dispersion efficiency as a function of both PEO and PPO molecular weights is more limited. Nevertheless, several observations can be made, including the fact that Tetronics 1107 and 1307 are found to be the most effective dispersing agents of all the copolymers studied. Despite their morphological differences compared with Pluronics, these Tetronics possess structures that fall within the optimal molecular weight window established by the Pluronics. The higher dispersion efficiencies measured overall for the Tetronics suggest that their ethylenediamine cores exhibit increased affinity for the graphene surface and promote exfoliation. Interestingly, Tetronic 304, which is the smallest molecular weight copolymer tested, displayed dispersion efficiencies comparable to much higher molecular weight copolymers such as Pluronic® F88 and Tetronic® 908. The PEO and PPO molecular weights of Tetronic® 304 place it well below the range of the molecular weights of the other Pluronic and Tetronic copolymers studied. Its comparatively high dispersion efficiency may result from a low barrier to intercalation during initial exfoliation, which successfully compensates for the reduced stabilization efficiency provided by its short PEO blocks, and/or fundamentally different dispersion behavior for block copolymers in this low-molecular-weight range.

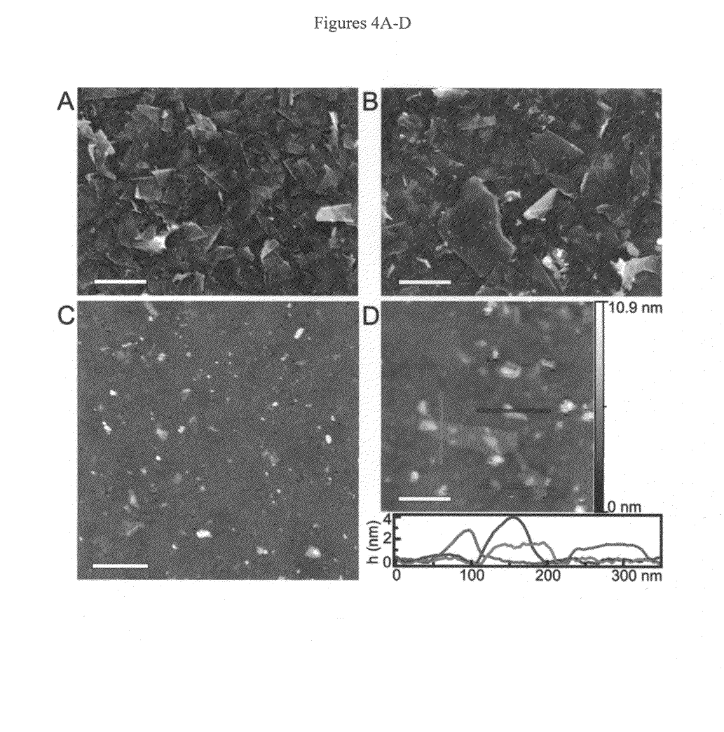

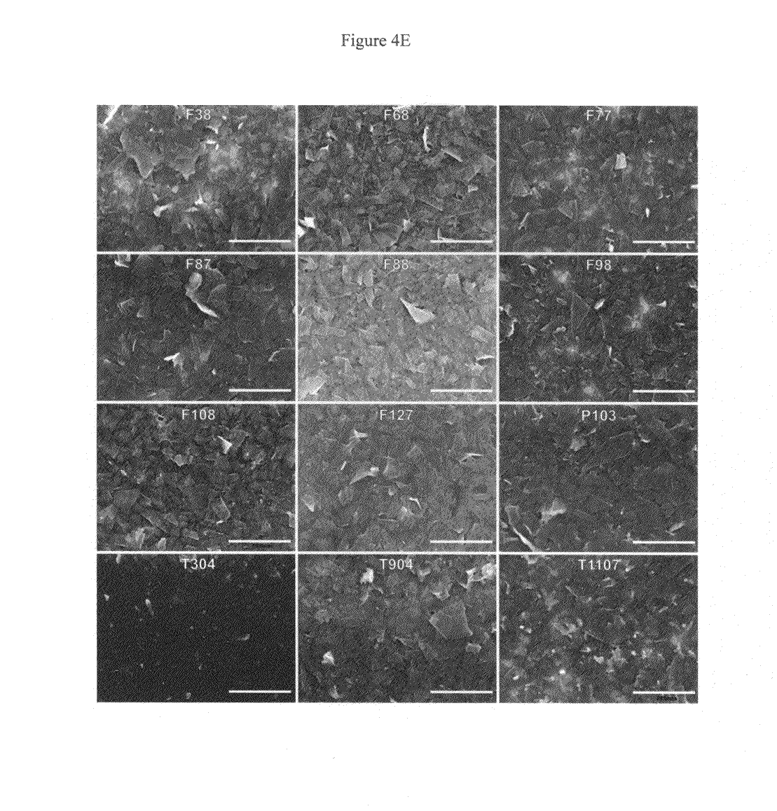

Thin films of restacked graphene were prepared from the graphene-copolymer dispersions using vacuum filtration. Following the transfer of these films to a suitable substrate, e.g., SiO₂, the graphene nanoplatelets were imaged using scanning electron microscopy (SEM). Representative SEM images of the graphene films obtained from Pluronic F77 and Tetronic 1107 are shown in FIGS. 4A-B. As illustrated in these images, the graphene nanoplatelets are deposited at random orientations in the plane parallel to the filtration membrane. The graphene nanoplatelets exhibit a wide distribution of surface areas, with most having lateral dimensions of a few hundred nanometers. SEM measurements of graphene samples prepared from various other copolymers showed similar distributions of platelet areas. (See FIG. 4E, with corresponding copolymer designation.) The exfoliated graphene was also deposited onto SiO₂-capped silicon wafers and imaged with AFM to assess nanoplatelet thickness (FIGS. 4C-D). The graphene thicknesses obtained from these measurements range from about 1 to about 4 nm, which is consistent with graphene nanoplatelets having 1 to ˜10 layers. The lateral dimensions of the graphene platelets in the AFM images range between ˜50 nm and several hundred nanometers.

Although the relatively small lateral areas of the graphene in, these dispersions are less than optimal for use in some high-performance electronic applications, such dimensions are comparable to graphene nanoplatelets produced using ionic surfactants under similar sonication conditions that have demonstrated competitive electronic conductivity in thin film form. Because sonication is known to reduce the size of solution-processed graphene, it is likely that the dimensions of copolymer-stabilized graphene can be increased by employing gentler sonication conditions over longer periods of time. However, larger area graphene platelets may actually be an impediment to biological applications by increasing cytotoxicity and inhibiting cellular uptake, thus suggesting that the relatively small area graphene available through this invention may possess advantages for biomedical applications.

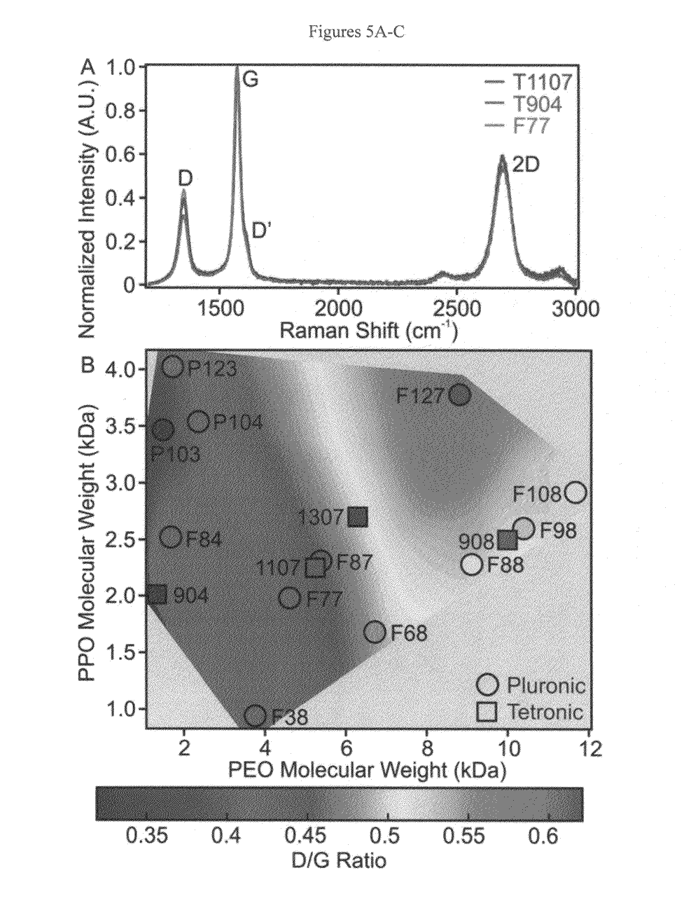

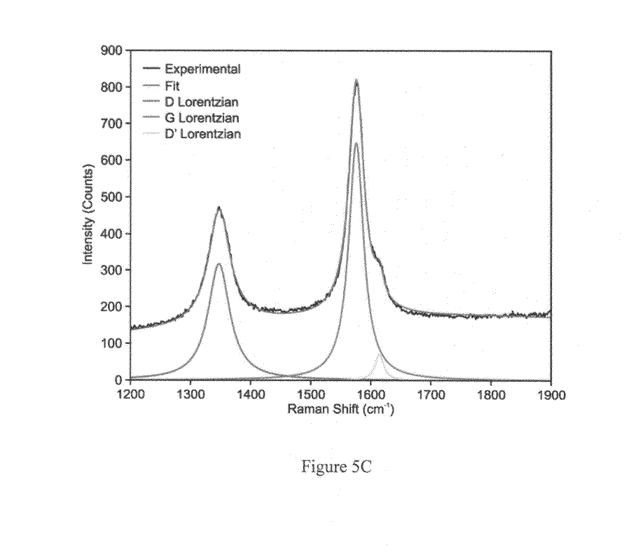

The thin films of graphene nanoplatelets were also characterized using Raman spectroscopy. The Raman spectra from the samples at a 514 nm excitation wavelength display three dominant peaks, G, 2D (or G′), and D, commonly observed in graphene as well as the D′ peak visible as a high-frequency shoulder to the G band (FIG. 5A). (See, Dresselhaus, M. S.; Jorio, A.; Souza Filho, A. G.; Saito, R. Defect Characterization in Graphene and Carbon Nanotubes Using Raman Spectroscopy. Philos. Trans. R. Soc., A 2010, 368, 5355-5377.) The 2D peak of the graphene samples is adequately described by a single Lorentzian, which is consistent with graphene sheets restacked with random interlayer registration. (See, Faugeras, C.; Nerriere, A.; Potemski, M.; Mahmood, A.; Dujardin, E.; Berger, C.; de Heer, W. A. Few-Layer Graphene on SiC, Pyrolitic Graphite, and Graphene: A Raman Scattering Study. Appl. Phys. Lett. 2008, 92, 011914.) The defect-related D and D′ peaks are significant in all copolymer-dispersed graphene samples. These defects are present at the edges of the small graphene nanoplatelets and are likely introduced to the graphene basal plane during horn ultrasonication.

To assess statistically the variations in defect density as a function of copolymer composition, Raman spectra of the films were taken at a minimum of eight different locations. The G, D, D′, and 2D peaks of the resulting spectra were fit to single Lorentzian lineshapes. (See FIG. 5C.) Analysis of these data revealed a general trend of increasing defect density (D/G ratio) of the graphene platelets for Pluronic copolymers of increasing molecular weight having hydrophilic domains larger than 3 kDa (FIG. 5B). The observed molecular weight dependence may be due to steric effects that hinder exfoliation by the bulkier, high-molecular-weight copolymers, which in turn lead to higher energies applied to the graphene as it is exfoliated. In contrast, the Tetronic dispersed graphene did not exhibit a correlation between molecular weight and defect density. These dispersing agents displayed lower defect densities overall, which can likely be understood by the improved exfoliation efficiency provided by their amine centers.

As demonstrated below, the methodologies of this invention can incorporate ultracentrifugation techniques to separate one or more fractions from a graphene dispersion. With respect to such techniques, it should be understood that isolating a separation fraction typically provides complex(es) formed by the surface active component(s) and graphene, whereas post-isolation treatment, e.g., removing the surface active component(s) from the graphene such as by washing, dialysis and/or filtration, can provide substantially pure or bare graphene. However, as used herein for brevity, reference may be made to graphene, graphene platelets or a dispersion thereof rather than the complexes and such reference should be interpreted to include the complexes as understood from the context of the description unless otherwise stated that non-complexed graphene is meant. As used herein, a separation fraction refers to a separation fraction that includes a majority of or a high concentration or percentage of graphene of a certain thickness or within a range of thickness dimensions. For example, a separation fraction can be enriched to include a higher concentration or percentage of graphene platelets with a thickness dimension less than about 10 nm—a concentration higher than that of the dispersion from which it was isolated.

Upon sufficient centrifugation (i.e., for a selected period of time and/or at a selected rotational rate at least partially sufficient to separate the graphene along the medium gradient), at least one separation fraction can be separated from the medium. Such fraction(s) can be isopycnic at a position along the gradient. An isolated fraction can include substantially monodisperse graphene platelets, for example, in terms of thickness dimensions. Various fractionation techniques can be used, including but not limited to, upward displacement, aspiration (from meniscus or dense end first), tube puncture, tube slicing, cross-linking of gradient and subsequent extraction, piston fractionation, and any other fractionation techniques known in the art.

The medium fraction and/or graphene fraction collected after one separation can be sufficiently selective in terms of separating the graphene by thickness dimension. However, in some embodiments, it can be desirable to further purify the fraction to improve its selectivity. Accordingly, in some embodiments, methods of the present teachings can include iterative separations. Specifically, an isolated fraction can be provided in composition with the same surface active component system or a different surface active component system, and the composition can be contacted with the same fluid medium or a different fluid medium, where the fluid medium can form a density gradient that is the same or different from the fluid medium from which the isolated fraction was obtained. In certain embodiments, fluid medium conditions or parameters can be maintained from one separation to another. In certain other embodiments, at least one iterative separation can include a change of one or more parameters, such as but not limited to, the identity of the surface active component(s), medium identity and/or formed medium density gradient with respect to one or more of the preceding separations. Accordingly, in some embodiments of the methods disclosed herein, the choice of the surface active component can be associated with its ability to enable iterative separations.

### EXAMPLES OF THE INVENTION

The following non-limiting examples and data illustrate various aspects and features relating to the methods, systems and compositions of the present invention, including the preparation of stable high-concentration graphene dispersions, as can be accomplished through the methodologies described herein. In comparison with the prior art, the present methods, systems and compositions provide results and data which are surprising, unexpected and contrary thereto. While the utility of this invention is illustrated through the use of representative block copolymeric components, it would be understood by those skilled in the art that comparable results are obtainable with various other surface active block copolymeric components, as are commensurate with the scope of this invention.

### Example 1

### Sonication

600 mg±5 mg of natural graphite flakes (Asbury Carbons, 3061 grade) were added to 8 mL of 1% w/v Pluronic® or Tetronic® aqueous solution inside a 15-mL-capacity, conical bottom plastic vial. This mixture was then sonicated for 30 minutes using a horn ultrasonicator equipped with a 3-mm-diameter probe (Fisher Scientific Model 500 Sonic Dismembrator). During this process, the sample vial was chilled in an ice/water bath, and sonication power was maintained at 16-18 W to ensure reliable comparisons between samples. Large initial loadings of graphite were used to maximize the concentrations of graphene exfoliated given the low cost of graphite flakes (˜$0.02 per gram).

### Example 2

### Centrifugation and Decantation

The sonicated graphene/graphite slurry was then centrifuged to eliminate poorly dispersed graphitic materials. The slurry was transferred to 1.5 mL centrifuge tubes and spun in an Eppendorf Model 5424 Microcentrifuge using a 45° fixed-angle (Rotor #: FA-45-24-11). The top 1 mL of graphene suspension, corresponding to a maximum sedimentation distance of approximately 1 cm, was carefully extracted from the centrifuge tubes following centrifugation. Four different centrifugation conditions were employed for each of the block copolymers studied and are listed in Table 1, below. Dispersions obtained using 5 minutes of centrifugation at 15,000 rpm were used for all the data presented. Dispersions prepared using weaker centrifugation conditions produced excessive levels of poorly-dispersed graphitic material while the stronger centrifugation condition pelleted a large proportion of the well-dispersed graphene.

TABLE 1
Centrifugation Processing Parameters

| Centrifugation | Centrifugation | Maximum Relative | Relative |
|---|---|---|---|
| Time (min) | Speed (rpm) | Centrifugal Force (g) | (speed)²(time) |
| 10 | 750 | 55 | 1 |
| 5 | 5000 | 2460 | 22.2 |
| 5 | 15,000 | 22,130 | 200 |
| 60 | 15,000 | 22,130 | 2400 |

### Example 3

### Concentration Characterization

The concentrations of the graphene dispersions were determined using optical absorbance spectroscopy. Measurements were conducted with a Cary 5000 spectrophotometer (Varian, Inc.) operating in dual beam mode. To ensure samples were measured in the linear response range of the spectrophotometer, the graphene dispersions were typically diluted by factors of 10 to 100 into 1% w/v aqueous solutions of the host block copolymer prior to absorbance acquisition. A reference sample containing 1% w/v of the Pluronic® or Tetronic® copolymer of interest was subtracted from the sample absorbance to compensate for its contribution to the absorbance spectrum. The resulting graphene concentrations and absorbance values determined for the undiluted dispersions are listed for all the block copolymers studied in Table 2. As discussed above, an extinction coefficient of 6600 L g⁻¹ m⁻¹ was employed for this analysis. Repeated experiments revealed a ˜6% uncertainty in the concentration measurements as a result of small changes in sonicator probe positioning and contamination of the supernatant by graphite weakly bound to the walls of the centrifuge tube during centrifugation.

TABLE 2
Concentration and Absorbance of Graphene Dispersed in Block Copolymers

| | | Molecular Weight (Da) | | | Concentration (g mL⁻¹)* | | | | OD/cm at λ = 660 nm* | | | |
|---|---|---|---|---|---|---|---|---|---|---|---|---|
| Polymer | | Total | PEO | PPO | A | B | C | D | A | B | C | D |
| Pluronics | F108 | 14600 | 11680 | 2920 | 1.078 | 0.225 | 0.049 | 0.016 | 71.2 | 14.9 | 3.23 | 1.03 |
| | F127 | 12600 | 8820 | 3780 | 1.255 | 0.303 | 0.064 | 0.014 | 82.9 | 20.0 | 4.25 | 0.91 |
| | F38 | 4700 | 3760 | 940 | 0.645 | 0.153 | 0.063 | 0.014 | 42.6 | 10.1 | 4.17 | 0.95 |
| | F68 | 8400 | 6720 | 1680 | 1.598 | 0.321 | 0.077 | 0.021 | 105.4 | 21.2 | 5.08 | 1.41 |
| | F77 | 6600 | 4620 | 1980 | 1.624 | 0.330 | 0.071 | 0.019 | 107.2 | 21.8 | 4.71 | 1.29 |
| | F87 | 7700 | 5390 | 2310 | 1.553 | 0.315 | 0.074 | 0.020 | 102.5 | 20.8 | 4.91 | 1.31 |
| | F88 | 11400 | 9120 | 2280 | 0.959 | 0.250 | 0.067 | 0.017 | 63.3 | 16.5 | 4.45 | 1.11 |
| | F98 | 13000 | 10400 | 2600 | 1.288 | 0.181 | 0.064 | 0.016 | 85.0 | 12.0 | 4.24 | 1.08 |
| | L62 | 2500 | 500 | 2000 | 0.098 | 0.014 | 0.001 | 0.001 | 6.46 | 0.9 | 0.05 | 0.06 |
| | L64 | 2900 | 1160 | 1740 | 0.011 | 0.003 | 0.000 | 0.000 | 0.7 | 0.2 | 0.03 | 0.01 |
| | P103 | 4950 | 1485 | 3465 | 1.040 | 0.136 | 0.026 | 0.004 | 68.6 | 9.0 | 1.71 | 0.29 |
| | P104 | 5900 | 2360 | 3540 | 1.136 | 0.181 | 0.045 | 0.011 | 75.0 | 11.9 | 2.95 | 0.73 |
| | P123 | 5750 | 1725 | 4025 | 0.751 | 0.108 | 0.024 | 0.005 | 49.6 | 7.1 | 1.56 | 0.35 |
| | P84 | 4200 | 1680 | 2520 | 1.053 | 0.203 | 0.043 | 0.008 | 69.5 | 13.4 | 2.85 | 0.50 |
| Tetronics | 304 | 1650 | 660 | 990 | 0.755 | 0.259 | 0.068 | 0.010 | 49.8 | 17.1 | 4.47 | 0.65 |
| | 904 | 6700 | 2680 | 4020 | 1.310 | 0.290 | 0.038 | 0.008 | 86.5 | 19.1 | 2.52 | 0.51 |
| | 908 | 25000 | 20000 | 5000 | 1.627 | 0.360 | 0.069 | 0.017 | 107.4 | 23.7 | 4.58 | 1.10 |
| | 1107 | 15000 | 10500 | 4500 | 1.752 | 0.396 | 0.086 | 0.023 | 115.7 | 26.1 | 5.69 | 1.54 |
| | 1307 | 18000 | 12600 | 5400 | 1.719 | 0.410 | 0.084 | 0.023 | 113.4 | 27.1 | 5.55 | 1.54 |

*A, B, C, D specify different centrifugation conditions of 10 minutes at 0.75 krpm, 5 minutes at 5 krpm, 5 minutes at 15 krpm, and 60 minutes at 15 krpm, respectively.

### Example 4

### Data Processing Used in FIGS. 3C and 5B

Two-dimensional color maps of the graphene concentrations as a function of Pluronic®/Tetronic® PEO and PPO molecular weights were obtained using Matlab. Experimental concentration values were first interpolated over a two-dimensional grid using the function grid data. These data were then smoothed by taking the moving average over an area within 500 Da of each PEO and PPO value.

### Example 5

### SEM Imaging of Graphene Films

The graphene films of FIG. 4A-E were imaged using a Hitachi 4800 SEM.

### Example 6

### AFM Imaging of Graphene

Individual graphene nanoplatelets were deposited onto SiO₂-capped Si wafers as described previously and annealed for 60 minutes at 250° C. (See, Sun, X. M.; Liu, Z.; Welsher, K.; Robinson, J. T.; Goodwin, A.; Zaric, S.; Dai, H. J. Nano-Graphene Oxide for Cellular Imaging and Drug Delivery. Nano Res. 2008, 1, 203-212). Measurements were performed using a Thermo Microscopes Autoprobe CP-Research AFM operating in tapping mode with conical probes (MikroMasch, NSC36/Cr—Au BS).

### Example 7

### Raman Spectroscopy of Graphene Films

Randomly oriented graphene films were prepared using vacuum filtration and transferred to receiving substrates as described in Green et al., supra. Raman spectroscopy was performed using a Renishaw in Via Raman Microscope at an excitation wavelength of 514 nm. G, D, D′, and 2D Raman peaks were fit to single Lorentzian lineshapes as shown in FIG. 5C with spectral background represented using a polynomial function. Statistically significant variations in the positions and widths of the Raman peaks were not observed as a function of the block copolymer. Likewise, variations in the 2D/G intensity ratio were not statistically significant.

### Example 8

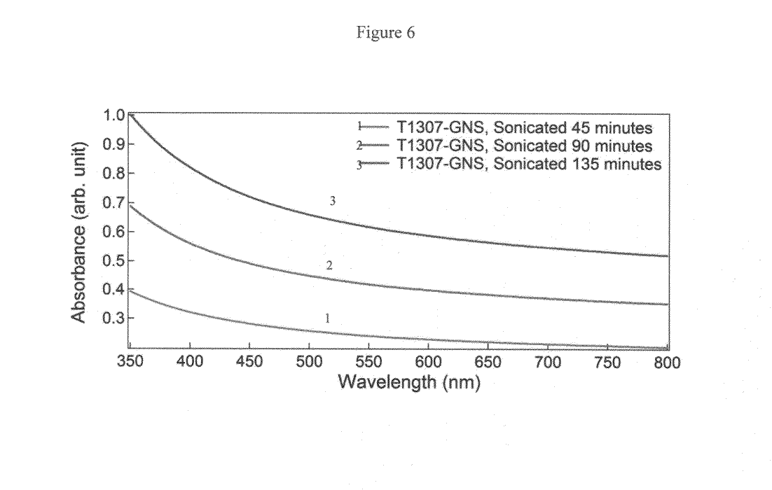

With reference to the data of Table 2, above, the Tetronic class of block copolymers, in particular T1307 and T1107, exhibited superior dispersion capacity as compared to Pluronic copolymers. The dispersion capacity of such surfactants can be further extended by increasing the ultrasonication time (FIG. 6). Following the Beer-Lambert law, the optical density from the absorbance spectrum can be used to deduce relative graphene concentration in solution. Such results show that Tetronic copolymers can further exfoliate and suspend higher graphene nanoplatelet concentrations than previously reported.

### Example 9

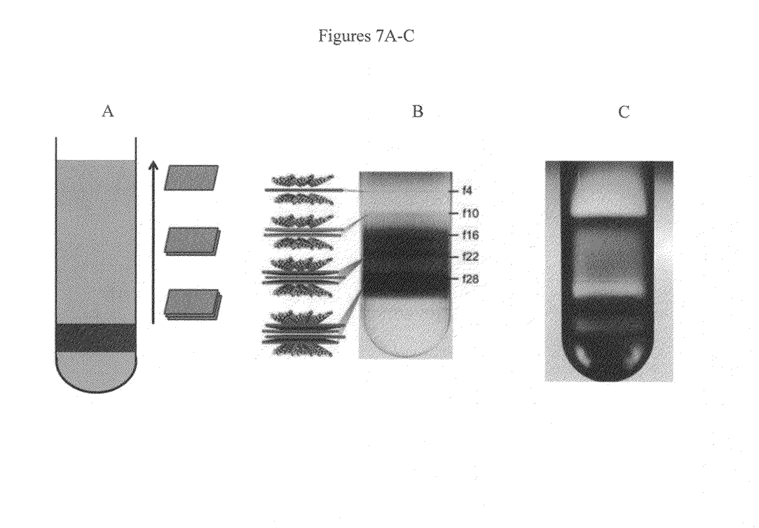

The compatibility of block copolymer-dispersed graphene nanoplatelets with density gradient ultracentrifugation (DGU) was considered. Previously, DGU was utilized to separate ionic surfactant-dispersed platelets by their layer number. (See, e.g., Green, A. A.; Hersam, M. C. Solution Phase Production of Graphene with Controlled Thickness via Density Differentiation. Nano Letters 2009, 9, 4031-4036.) In that work, the nanoplatelets were encapsulated by sodium cholate, a commonly used anionic surfactant for DGU. However, sodium cholate is ionic, which leads to detrimental effects in biological systems. To avoid that issue, DGU was employed with Tetronic-encapsulated graphene nanoplatelets (FIG. 7). During DGU, the suspended surfactant-nanoplatelet complexes travel toward their isopycnic point, where their buoyant densities match those of the density gradient medium. The success of DGU can be observed through the visible formation of discrete bands of the suspended graphene nanoplatelets inside the ultracentrifuge tube, which indicates that the nanoplatelet complexes have been effectively separated according to buoyant density. The ultracentrifuge tube of T1307-complexed nanoplatelets after DGU shows a dark band on top of the density gradient, which contains the most buoyant graphene nanoplatelets, as demonstrated previously.

More specifically, six grams of natural graphite flakes (3061 grade material from Asbury Graphite Mills) were placed in 70 mL of 2% w/v T1307 aqueous solution inside a 120 mL capacity stainless steel beaker. This mixture was ultrasonicated using a Fisher Scientific Model 500 Sonic Dismembrator with a 13-mm diameter tip for one hour at 40% of the maximum amplitude. 32 mL of graphene dispersion was then placed on top of a 6 mL underlayer containing 60% w/v iodixanol (1.32 g/mL) and 2% w/v T1307. These step gradients were ultracentrifuged in an SW 32 rotor (Beckman Coulter) for 24 hours at 28 krpm at temperature of 22 C. Following ultracentrifugation, a 60% w/v iodixanol, 2% w/v T1307 displacement layer was slowly infused near the band of concentrated graphene to both separate it from precipitated materials below and to raise the position of the band in the centrifuge tube for more reliable fractionation. The concentrated material was then collected using a piston gradient fractionator (Biocomp Instruments).

Subsequently, the concentrated T1307-graphene dispersion was diluted to 4 mL of solution containing 46% w/v iodixanol, which was then placed under a 15 mL linear density gradient of 25-45% w/v iodixanol (1.13-1.24 g/mL). Below the graphene layer, a dense 6 mL underlayer of 60% w/v iodixanol was placed, and 0% w/v iodixanol aqueous solution was used to cap the ultracentrifuge tube above the linear density gradient. All solutions contained 2% w/v T1307. The prepared linear density gradients were ultracentrifuged in an SW 32 rotor for 24 hours at 28 krpm at temperature of 22 C. With reference to FIG. 7C, the graphene dispersion was separated by nanoplatelet thickness dimension, with fractions isopycnic at positions along the density gradient. The upper most buoyant fraction is collected as described above.

### Example 10

As understood by those in the art, aqueous iodixanol is a common, widely used non-ionic density gradient medium. However, other media can be used with good effect, as would also be understood by those individuals. More generally, any material or compound stable, soluble or dispersible in a fluid or solvent of choice can be used as a density gradient medium. A range of densities can be formed by dissolving such a material or compound in the fluid at different concentrations, and a density gradient can be formed, for instance, in a centrifuge tube or compartment. More practically, with regard to choice of medium, the graphene dispersion should also be soluble, stable or dispersible within the fluids/solvent or resulting density gradient. Likewise, from a practical perspective, the maximum density of the gradient medium, as determined by the solubility limit of such a material or compound in the solvent or fluid of choice, should be at least as large as the buoyant density of the graphene (and/or in composition with one or more surfactants) for a particular medium.

Accordingly, with respect to this invention, any aqueous or non-aqueous density gradient medium can be used providing the graphene is stable; that is, does not aggregate to an extent precluding useful separation. Alternatives to iodixanol include but are not limited to inorganic salts (such as CsCl, Cs₂SO₄, KBr, etc.), polyhydric alcohols (such as sucrose, glycerol, sorbitol, etc.), polysaccharides (such as polysucrose, dextrans, etc.), other iodinated compounds in addition to iodixanol (such as diatrizoate, nycodenz, etc.), and colloidal materials (such as but not limited to percoll). Other media useful in conjunction with the present invention would be understood by those skilled in the art made aware of this invention.

### Example 11

The significance of developing a facile preparation method for biocompatible graphene nanoplatelets has been verified. (See, e.g., Duch, M. C.; Budinger, G. R.; Liang, Y. T.; Soberanes, S.; Urich, D.; Chiarella, S. E.; Campochiaro, L. A.; Gonzalez, A.; Chandel, N. S.; Hersam, M. C.; Mutlu, G. M. Minimizing Oxidation and Stable Nanoscale Dispersion Improves the Biocompatibility of Graphene in the Lung. Nano Letters 2011, 11, 5201-5207.) The referenced study indicates that the pulmonary toxicity of graphene is minimized when administered in vivo as a dispersion with block copolymers of the sort described herein. (By contrast, aggregated graphene in water tends to block airways and induce local fibrotic response, while water-soluble graphene oxide increases mitochondrial oxidant generation and induces apoptosis in lung macrophages.) Thus, graphene nanoplatelets processed according to methods of this invention are considered promising candidates as drug delivery agents or imaging contrast agents in vivo.

As demonstrated, nonionic biocompatible block copolymers can be used to disperse pristine graphene at high concentrations in aqueous solution. Several such copolymers, Pluronic® F68, F77, F87 and Tetronic® 1107 and 1307, readily produce graphene suspensions with optical densities exceeding 4 OD cm 1 from the visible to the near infrared, corresponding to graphene concentrations exceeding about 0.07 mg mL⁻¹. The ease of processing and high dispersion efficiency of these copolymers suggests use with graphene in biomedical applications, particularly where the low cost and high surface area of graphene provide it with distinct advantages over competing nanomaterials.

We claim:

1. A method of preparing an aqueous graphene dispersion, said method comprising:
providing a composition comprising a graphitic composition comprising natural graphene, at least one nonionic surface active polymeric component and an aqueous medium;
sonicating said composition for at least one of a time and at an energy sufficient to exfoliate said graphene component and disperse said graphene component within said aqueous medium; and
centrifuging said sonicated composition for at least one of a time and a rotational rate to separate said dispersed graphene component from undispersed graphitic material.

2. The method of claim 1 wherein said polymeric component comprises a block copolymer selected from linear and X-shaped amphiphilic poly(alkylene oxide) block copolymers and combinations thereof.

3. The method of claim 2 wherein a said block copolymer comprises poly(ethylene oxide) blocks and poly(propylene oxide) blocks.

4. The method of claim 3 wherein said copolymer is linear, and the molecular weight of said poly(ethylene oxide) blocks is about 60-about 90 wt. % of said copolymer.

5. The method of claim 3 wherein said copolymer is X-shaped, and the molecular weight of said poly(ethylene oxide) blocks is about 30-about 90 wt. % of said copolymer.

6. The method of claim 5 wherein said molecular weight is about 70-about 80 wt. % of said copolymer.

7. The method of claim 1 wherein said centrifugation separates at least one fraction of said dispersed graphene component, said fraction enriched with graphene platelets of a thickness dimension, said enrichment relative to said dispersed graphene component.

8. The method of claim 7 comprising isolation of said separation fraction and repeating said centrifugation.

9. A method of using a surface active block copolymeric component to affect dispersion of graphene in an aqueous medium, said method comprising:
providing a composition comprising a graphene source material comprising a graphene component, at least one surface active block copolymer component comprising poly(alkylene oxide) blocks and an aqueous medium;
sonicating said composition for at least one of a time and at an energy sufficient to exfoliate said graphene component and disperse said graphene component within said aqueous medium; and
centrifuging said sonicated composition for at least one of a time and a rotational rate to separate said dispersed graphene component from undispersed graphitic material.

10. The method of claim 9 wherein a said block copolymer comprises poly(ethylene oxide) blocks and poly(propylene oxide) blocks.

11. The method of claim 10 wherein said copolymer is linear, and the molecular weight of said poly(ethylene oxide) blocks is about 60-about 90 wt. % of said copolymer.

12. The method of claim 10 wherein said copolymer is X-shaped, and the molecular weight of said poly(ethylene oxide) blocks is about 30-about 90 wt. % of said copolymer.

13. The method of claim 12 wherein said molecular weight is about 70-about 80 wt. % of said copolymer.

14. A method of using a density gradient to separate graphene platelets, said method comprising;
providing a composition comprising a graphene source material comprising a graphene component, at least one surface active block copolymer component comprising poly(ethylene oxide) and poly(propylene oxide) blocks and an aqueous medium;
sonicating said composition for at least one of a time and at an energy sufficient to exfoliate said graphene component and disperse said graphene component within said aqueous medium, said dispersed graphene component comprising platelets varied by thickness dimension;
contacting a said dispersed graphene component with a fluid medium comprising a density gradient, and centrifuging said dispersed graphene component for at least one of a time and a rotational rate at least partially sufficient to induce a graphene buoyant density approximating a density along said gradient and concentrating at least a portion of said graphene dispersion therein; and
separating said concentrated graphene dispersion into at least one separation fraction enriched with graphene platelets of a thickness dimension, said enrichment relative to said composition dispersion.

15. The method of claim 14 wherein said copolymer is linear, and the molecular weight of said poly(ethylene oxide) blocks is about 60-about 90 wt. % of said copolymer.

16. The method of claim 14 wherein said copolymer is X-shaped, and the molecular weight of said poly(ethylene oxide) blocks is about 30-about 90 wt. % of said copolymer.

17. The method of claim 16 wherein said molecular weight is about 70-about 80 wt. % of said copolymer.

18. The method of claim 14 wherein said fluid medium comprises a plurality of aqueous iodixanol concentrations, said density gradient comprising a range of concentration densities.

19. The method of claim 18 wherein a fraction of said graphene dispersion is isopycnic at a position along said density gradient.

20. The method of claim 14 wherein a said separation fraction is administered in vivo.

21. A graphene composition comprising graphene platelets complexed with an ethylene diamine cross-linked poly(ethylene oxide)-poly(propylene oxide) block copolymer, said composition in an aqueous medium.

22. The composition of claim 21 wherein the molecular weight of said poly(ethylene oxide) blocks is about 30-about 90 wt. % of said copolymer.

23. The composition of claim 22 wherein said molecular weight is about 70-about 80 wt. % of said copolymer.

24. The composition of claim 21 wherein the concentration of said complex is greater than about 0.07 mg mL⁻¹.

25. The composition of claim 21 administered in vivo.
